(19) United States
(12) Patent Application Publication
(10) Pub. No.: US 2006/0212072 A1
(43) Pub. Date: Sep. 21, 2006

(54) Polyolefin sutures having enhanced durability

(76) Inventors: Brian J. Cuevas, Middletown, CT (US); Frank R. Schiretz JR., Middletown, CT (US); Matthew D. Cohen, Berlin, CT (US); Joseph Hotter, Middletown, CT (US)

Correspondence Address: UNITED STATES SURGICAL,;A DIVISION OF TYCO HEALTHCARE GROUP LP, 195 MCDERMOTT ROAD, NORTH HAVEN, CT, 06473, US

(21) Appl. No.: 11/081,390

(22) Filed: Mar. 16, 2005

(51) Int. Cl. A61L 17/00

(52) U.S. Cl. 606/228

(57) ABSTRACT

Polyolefin sutures include a fray reducing amount of a thiodipropionate ester, and optionally a fatty acid diester, a synthetic wax and/or a pigment.

### TECHNICAL FIELD

The present disclosure relates to surgical sutures, and particularly to a polyolefin suture having improved durability and fray resisting characteristics.

### DESCRIPTION OF RELATED ART

Polyolefin sutures are known in the art. Such sutures are non-absorbable and generally include polypropylene or polymeric combinations of ethylene and propylene. The polymeric components of the polyolefin sutures are generally melt spun to produce filaments for use in fabricating the surgical suture strands. Polypropylene sutures can be advantageously produced as monofilament sutures.

Various methods are known for making polypropylene sutures. For example, U.S. Pat. No. 5,217,485 to Liu et al. discloses a process for making a polypropylene monofilament suture by melt extruding the monofilament, stretching the solidified monofilament, then allowing the monofilament to equilibrate, or “rest”, prior to annealing.

Polypropylene monofilament sutures are known to exhibit a limited amount of fraying as the suture passes over itself, e.g., when tying knots. Polypropylene sutures have thus become the preferred suture for cardiovascular and vascular surgery.

While the limited amount of fraying exhibited by polypropylene monofilament sutures does not substantially hamper the performance of the suture, there remains room for improvement in the processing and handling characteristics of such sutures.

### SUMMARY

Sutures having improved handling characteristics are made in accordance with the disclosure from a composition containing a polyolefin and a fray reducing amount of a thiodipropionate of the formula
R—OOCCH₂CH₂SCH₂CH₂COO—R,
where R is a C₁₃ or greater alkyl group. The compositions optionally include a fatty acid diester, a synthetic wax and a dye.

Methods for producing sutures from such compositions include the steps of extruding a composition containing a polyolefin thiodipropionate and, optionally, a fatty acid diester, a synthetic wax and a dye.

### BRIEF DESCRIPTION OF THE FIGURES

FIG. 1 is a diagram of a system utilized for testing the fray resistance of sutures of the present disclosure.

FIG. 2 is a graph depicting cycle to break data of sutures of the present disclosure having varying concentrations of distearylthiodipropionate (DSTDP) and a constant concentration of polyethylene glycol distearate.

FIG. 3. is a graph depicting cycle to break data of sutures of the present disclosure having varying concentrations of polyethylene glycol distearate and a constant concentration of DSTDP.

FIG. 4A and FIG. 4B. are graphs depicting cycle to break data and tensile strength data, respectively, of sutures of the present disclosure having DSTDP, polyethylene glycol distearate, polyethylene wax, and dye, compared with two control sutures.

### DETAILED DESCRIPTION

Sutures in accordance with the present disclosure include a polyolefin in combination with a fray reducing amount of a thiodipropionate. In some embodiments, the sutures of the present disclosure may also contain a fray reducing amount of a fatty acid diester, a synthetic wax, or both.

All composition percentages listed herein shall be understood to be by weight unless otherwise indicated. All quantities set forth below, except in the claims, shall be understood to be modified by the term “about”.

Any suitable polyolefin known in the art may be used in producing a suture in accordance with their disclosure. Polyolefins which may be utilized include, for example, polyethylene, polypropylene and copolymers of polyethylene and polypropylene. Blends of polyolefins, (e.g. blends of polyethylene and polypropylene) may also be used. In particularly useful embodiments, polypropylene, which can be isotactic polypropylene or a mixture of isotactic and syndiotactic or atactic polypropylene, is used to form the suture. Crystalline polypropylenes of from about 150,000 to about 500,000 average molecular weight (Mw) can be especially useful. Suitable isotactic polypropylene resins include those possessing a weight average molecular weight (Mw) of from about 200,000 to about 350,000, a number average molecular weight (Mn) of from about 50,000 to about 180,000, and a calculated dispersity (Mw/Mn) of from about 2.0 to about 4.0. The isotactic polypropylene resins can advantageously possess a melt flow index in g/10 min of from about 2 to about 6 and typically from about 3.5 to about 4.5. Isotactic polypropylene resins are readily available from numerous commercial sources.

Thiodipropionates suitable for use herein include a propanoic ester backbone, in which the functional side chains are extended with aliphatic groups in place of a hydrogen atom. The general molecular structure of particularly useful thiodipropionates is shown below:
R—OOCCH₂CH₂SCH₂CH₂COO—R
where R is a C₁₃ or greater alkyl group.

In certain embodiments, the thiodipropionate used to form the polyolefin suture as described herein is a thiodipropionate such as 3,3′-thiobis-,dioctadecyl ester propanoic acid, also known as distearylthiodipropionate (DSTDP), or 3,3′-thiobis-,ditridecyl ester propanoic acid, also known as ditridecylthiodipropionate (DTTDP), and combinations thereof. In some embodiments, 3,3′-thiobis-, didodecyl ester propanoic acid, also known as dilaurylthiodipropionate (DLTDP), may be combined with the thiodipropionate having at least 32 carbon atoms. In a particularly useful embodiment, the thiodipropionate used to form the polyolefin suture can be a distearylthiodipropionate such as CARSTAB® DSTDP or MORSTILLE® DSTDP, both sold by Struktol (Stow, Ohio).

While thiodipropionates such as DSTDP have been previously used with polyolefins as antioxidants, in the present disclosure the thiodipropionates are utilized to enhance the fray resisting characteristics of sutures. Without wishing to be bound by any theory, it is believed the thiodipropionates, when mixed with a polyolefin at appropriate concentrations, undergo exudation, i.e., they bloom to the outer surfaces of the polyolefin suture. Once exuded to the outer surfaces of the suture, the thiodipropionates can act as a lubricant and significantly improve the fray resisting characteristics of the polyolefin suture during the suturing process.

The thiodipropionate can be present in the suture in amounts ranging from about 0.01% to about 1.5% by weight of the suture, typically from about 0.1% to about 1.0% by weight of the suture. In particularly useful embodiments, the thiodipropionate can represent from about 0.2% to about 0.6% by weight of the suture.

In some useful embodiments, a fray reducing amount of a fatty acid diester may be added to the suture of the present disclosure. Any fatty acid diester known to one skilled in the art can be used in forming the suture described in the present disclosure. Suitable fatty acids include C₁₀-C₂₆ fatty acids such as stearic, lauric, palmitic, myristic, arachidic, behenic, and similar acids. In some embodiments the fatty acid diester is a diester of a polyalkylene glycol. Suitable polyalkylene glycols include C₂-C₆ alklyene glycols, such as polyethylene glycol and polypropylene glycol.

In particularly useful embodiments, the fatty acid diester can be a fatty acid diester of polyethylene glycol such as, for example, polyethylene glycol distearate (PEG distearate). In particular, a suitable PEG distearate for use in the method described herein has a melting point of from about 35° C. to about 37° C., an acid value of about 5.0, an iodine value of about 0.41, and a saponification value of about 117.0. Suitable PEG distearates are readily available from numerous commercial sources.

Where utilized, the fatty acid diester can be present in the suture in amounts ranging from about 0.01% to about 5.0% by weight of the suture. In some embodiments, the fatty acid diester can represent from about 0.1% to about 2.0% by weight of the suture. In particularly useful embodiments, the fatty acid diester can represent from about 0.2% to about 1.5% by weight of the suture.

Sutures produced in accordance with the present disclosure may also have a synthetic wax included therein. Suitable synthetic waxes for use in the present disclosure include polyethylene wax, ethylene copolymer wax, and halogenated hydrocarbon waxes. In some embodiments, the synthetic wax can be a polyethylene wax, such as a low molecular weight polyethylene wax. As used herein, the term “polyethylene wax” refers to both ethylene homopolymers and copolymers of ethylene with α-olefins having a chain length of C₃-C₁₈, each having a melt viscosity measured at 140° C. of from about 5 to about 20000 mPa·sec. In certain embodiments, the polyethylene wax is a low molecular weight polyethylene wax having a weight average molecular weight in the range of from about 500 to about 4000 Daltons, typically from about 1000 to about 3000 Daltons, more typically about 1500 to about 2500 Daltons. The polyethylene wax can also be a low density polyethylene, a linear low density polyethylene, a non-linear low density polyethylene, or a high density polyethylene. As used herein, “LDPE” is a low density polyethylene, usually branched, having a density from about 0.91 to about 0.94 g/cc; “HDPE” is a high density polyethylene having a density above about 0.95 g/cc; and “LLDPE” is a linear low density polyethylene having a density of about 0.91 to about 0.95 g/cc. Suitable polyethylene waxes are readily available from numerous commercial sources.

Where utilized, the synthetic wax, such as a polyethylene wax, can be present in the suture in amounts ranging from about 0.01% to about 2.0% by weight of the suture. In some embodiments, the synthetic wax can represent from about 0.1% to about 1.5% by weight of the suture. In a particularly useful embodiment, the synthetic wax can represent from about 0.2% to about 1.0% by weight of the suture.

In some embodiments a synergistic effect may be observed in sutures containing a fray reducing amount of both a thiodipropionate and a fatty acid diester. Such sutures may be found to possess significantly improved fray resisting characteristics compared to either additive alone. The addition of a synthetic wax such as a polyethylene wax may further reduce the fray resistance characteristics of the resulting suture.

In some embodiments, it may be desirable to include a pigment in the composition of the present disclosure, thereby providing color to the sutures of the present disclosure. The term “pigment” herein is used interchangeably with the term “dye” and refers to such compounds or particles that absorb visible and/or infrared light. Suitable pigments for dyeing polypropylene filaments are known to those skilled in the art. Such pigments include, but are not limited to, carbon black, bone black, copper phthalocyanine dyes, D&C Green No. 6, and D&C Violet No. 2 as described in the handbook of U.S. Colorants for Food, Drugs and Cosmetics by Daniel M. Marrion (1979). Other dyes which may be used include indocyanine green, methylene blue, fluorescein, india ink, Prussian blue, eosins, acridine, iron oxide, and acramine yellow. Those skilled in the art will recognize that detectable moieties may also be utilized with such dyes. Such detectable moieties include, but are not limited to, fluorescers, bioluminescent and chemiluminescent molecules, and the like.

Where utilized, sutures in accordance with the present disclosure may be dyed by adding up to about 0.1% to about 1.0% (by weight of the final suture composition) dye, typically about 0.2% to about 0.6% dye to the resin composition prior to extrusion to form a suture. In one embodiment, a copper phthalocyanine dye such as beta phthalocyaninato (2-) copper dye may be used. Such dyes are readily available from numerous commercial sources and, in some embodiments, may be obtained in a form which includes polyolefins such as polyethylene.

Sutures of the present disclosure may be produced by melt extrusion or “spinning” of the polyolefin combined with the fray reducing amount of the thiodipropionate, optionally in combination with the fray reducing amount of the fatty acid diester, synthetic wax, and/or dye. An exemplary process for manufacturing a suture is described in U.S. Patent Application Pub. No. 2002/0177876, the contents of which are incorporated by reference herein.

In one embodiment, the first step in producing sutures of the present disclosure can be performed by directly adding the thiodipropionate to the polyolefin, either prior to or during melting. In some embodiments, the thiodipropionate may be combined with the polyolefin by dry blending. The resulting blend will possess thiodipropionate in an amount as noted above, i.e., an amount ranging from about 0.01% to about 1.5% by total weight of the composition, typically from about 0.1% to about 1.0% by total weight of the composition, more typically from about 0.2% to about 0.6% by total weight of the composition.

In other embodiments, a composition to be extruded may be prepared by first pre-blending a first portion of polyolefin and thiodipropionate to make what is commonly referred to as a “master batch”, which is then combined with a second portion of polyolefin to produce a batch having the desired weight percentages of polyolefin and thiodipropionate.

The amount of thiodipropionate added to the master batch of preblended polypropylene (in pellet or other suitable form) is often referred to as the “letdown” or “letdown ratio”. As those skilled in the art will appreciate, the ratio of additional polyolefin to the preblended masterbatch can be adjusted to produce a second batch, and thus a product, having any target percentage of thiodipropionate. In some cases mixing a small quantity of pre-blended polyolefin/thiodipropionate with standard polyolefin pellets may result in better dispersion of the thiodipropionate in the second batch, which may then be heated to form the subsequent polymer melt. The preblended polyolefin can be produced at one facility or operation and formed into a master batch of pellets which can then be stored and/or transferred to the suture fabrication operation. The polyolefin used to make the pre-blended batch of polyolefin/thiodipropionate can have the same characteristics (e.g., molecular weight, melt flow index, etc.) as the standard polyolefin with which the pre-blended batch is combined to form the second batch, which is then heated to form a polymer melt.

When a specific amount or percentage of thiodipropionate is needed to achieve a suture of the present disclosure, a masterbatch can be prepared and then can be “let down” to obtain the desired amount of thiodipropionate in the resulting suture. For example, if the desired thiodipropionate level in the final suture fiber is 1.0% by weight based on the weight of the polyolefin, a 20% masterbatch, for example, can be let down. The term “let down,” as used herein, refers to the ratio of:
(w)(z)=(w′)(z′)
wherein

w is the amount of the masterbatch,

z is the percentage of thiodipropionate in the masterbatch,

w′ is the batch size of the polyolefin utilized to produce a suture (in the same units as w), and

z′ is the percentage of thiodipropionate in the polyolefin utilized to produce a suture of the present disclosure.

Therefore, in the above example, if w′ is 100 pounds, z′ is 1.0% and z is 20%, then 5 pounds of the masterbatch is needed to produce a suture having a thiodipropionate concentration of 1.0%. Thus, in this example, the let down ratio would be 5:1, which can also be referred to as a 5% let down.

Where the polyolefin is prepared by the masterbatch process described above, the let down can range from about 5% to about 100%.

In another embodiment, the first step of the method can be similarly performed by adding both a fray reducing amount of a thiodipropionate and a fray reducing amount of a fatty acid diester to the polyolefin, either prior to or during melting. As described above, in some embodiments it may be advantageous to first form a master batch of preblended polyolefin containing a first portion of polyolefin, thiodipropionate, and fatty acid diester, and then combine the master batch with a second portion of polyolefin to obtain a second batch utilized to form a resin having the desired levels of polyolefin, thiodipropionate and fatty acid diester. In either case, the amount of thiodipropionate will be present in the resulting composition in the amount as noted above, i.e., it can thus range from about 0.01% to about 1.5% by weight of the total composition, typically from about 0.1% to about 1.0% by weight of the total composition, more typically from about 0.2% to about 0.6% by weight of the total composition. The amount of fatty acid diester will also range in the amount noted above, i.e., from about 0.01% to about 5.0% by weight of the total composition, typically from about 0.1% to about 2.0% by weight of the total composition, more typically from about 0.2% to about 1.5% by weight of the total composition.

Dyes or pigments may be added as described above to any of the resin compositions described herein to enhance visualization of the resulting suture in the surgical field.

In still another embodiment, the first step of the method can be similarly performed by adding a fray reducing amount of a thiodipropionate, a fray reducing amount of a fatty acid diester, a synthetic wax, and optionally a dye to the polyolefin, either prior to or during melting. Here also, as described above, in some embodiments a mixture of polyolefin, thiodipropionate, fatty acid diester, synthetic wax, and dye, if any, may be prepared by making a master batch of preblended polyolefin containing a first portion of polyolefin, a thiodipropionate, a fatty acid diester, a synthetic wax, and optionally a dye. The master batch may then be mixed with a second portion of standard polyolefin pellets to provide a second batch which may then be utilized to form a suture having the overall desired level of thiodipropionate, fatty acid diester, synthetic wax, and dye, in amounts noted above. The ratio of standard polyolefin to the preblended polyolefin can be adjusted to produce a product having any target percentage composition of thiodipropionate, fatty acid diester, synthetic wax and/or dye. Mixing a small quantity of pre-blended polyolefin with standard polyolefin pellets may achieve better dispersion of the thiodipropionate, fatty acid diester, synthetic wax and/or dye in the subsequent polymer melt than direct addition of thiodipropionate, fatty acid diester, synthetic wax and/or dye to the polyolefin.

The next step in producing sutures in accordance with the present disclosure involves heating the polyolefin in combination with the thiodipropionate, optionally in combination with the fatty acid diester, polyethylene wax, and/or dye, to form a polymer melt. This melt is then extruded and cooled to form a filament which can then be sent to further processing such as stretching. The melt contains substantially no water or organic solvents, and no substances which would be incompatible with body tissue.

In some embodiments, other additives may be included in the suture material. Suitable additives are known to those skilled in the art and include antioxidants, such as hindered phenols; or vitamin E; antistatic agents; antacids such as metal soaps, including calcium stearate; and additional thioesters, such as DLTDP.

In some embodiments, sutures of the present disclosure can be monofilament sutures. In other embodiments, sutures of the present disclosure can be multifilament sutures. When more than one filament is used to form a suture, the filaments may be braided, twisted, entangled, intertwined or arranged in some other multifilament configuration. Particularly useful braid structures for sutures are the braid structures described in U.S. Pat. Nos. 5,019,093 and 5,059,213, the disclosures of which are incorporated herein by reference.

Sutures as described herein can be used to secure tissue in a desired position. As seen in FIG. 5, a combined needle/suture 100 may be produced wherein the suture 101 may be attached to the surgical needle 102 by methods well known in the art.

Wounds may be sutured by approximating tissue and passing the needled suture through tissue to create wound closure. The needle is then typically removed from the suture and the suture tied.

The sutures and methods described herein are illustrated by the following non-limiting examples.

### EXAMPLE 1

Sample monofilament sutures, size 5/0, were prepared in accordance with the general procedures described above. The polymer melt was prepared by combining polypropylene with a polyethylene wax, polyethylene glycol distearate, a dye, and distearylthiodipropionate. The polypropylene possessed certain additives: 0.2% of a mixture of a hindered phenol antioxidant, calcium stearate, and an antistatic agent (Chemstat 273E), and 0.4% DLTDP. Details of the amounts of the above components in the various samples are set forth in Table 1 below.

TABLE 1

| | | | Polethlyene | | | | | | | |
|---|---|---|---|---|---|---|---|---|---|---|
| | Polypro | | Wax | | PEG Distearate | | DSTDP | | Dye | |
| Sample | % | g | % | g | % | g | % | g | % | G |
| 1 | 98.446 | 984.46 | 0.354 | 3.54 | 0.750^{C} | 7.50 | 0 | 0 | 0.450 | 4.50^{B} |
| 2 | 98.146 | 981.46 | 0.354 | 3.54 | 0.750 | 7.50 | 0.300 | 3.00 | 0.450 | 4.50^{B} |
| 3 | 97.596 | 975.96 | 0.354 | 3.54 | 1.000 | 10.00 | 0.600 | 6.00 | 0.450 | 4.50^{B} |
| 4 | 97.946 | 979.46 | 0.354 | 3.54 | 0.750 | 7.50 | 0.500 | 5.00 | 0.450 | 4.50^{B} |
| 5 | 98.046 | 980.46 | 0.354 | 3.54 | 0.750 | 7.50 | 0.400 | 4.00 | 0.450 | 4.50^{B} |
| 6 | 98.046 | 9804.60 | 0.354 | 35.40 | 0.750 | 75.00 | 0.400 | 40.00 | 0.450 | 45.00^{B} |
| 7 | 98.046 | 9804.60 | 0 | 0 | 0.750 | 75.00 | 0.400 | 40.00 | 0.804 | 80.40^{A} |
| 8 | 98.796 | 987.96 | 0 | 0 | 0 | 0 | 0.400 | 4.00 | 0.804 | 8.04^{A} |
| 9 | 98.796 | 987.96 | 0.354 | 3.54 | 0 | 0 | 0.400 | 4.00 | 0.450 | 4.50^{B} |

A = EUPOLEN ® Blue 70-9001
B = HELIOGEN ® Blue K 7090
C = PEG 6000 distearate

Sutures were then prepared from the above samples utilizing the meters in Table 2 below.

TABLE 2

| Parameter | Set Point |
|---|---|
| Barrel 1 Temp. (° C.) | 200 ± 5 |
| Barrel 2 Temp. (° C.) | 200 ± 5 |
| Barrel 3 Temp. (° C.) | 200 ± 5 |
| Clamp Temp (° C.) | 200 ± 5 |
| Adaptor Temp. (° C.) | 200 ± 5 |
| Block Temp. (° C.) | 200 ± 5 |
| Pump Temp. (° C.) | 200 ± 5 |
| Die Temp. (° C.) | 200 ± 5 |
| Aux. Die Temp. (° C.) | 210 ± 5 |
| Barrel (psi) | 450-3000 |
| Pump (psi) | 500 ± 200 |
| Quench (° C.) | 40 ± 5 |
| Godet 1 | 7.2 (+0.1, −0.2) |
| (meters/min, “mpm” | |
| Godet 2 mpm | 49.5 (±0.5) |
| Godet 3 mpm | 68.6 (±0.5) |
| Godet 4 mpm | 50.9 (+1.7, −1.8) |
| Draw 1 (° C.) | 115 ± 3 |
| Draw 2 (° C.) | 130 ± 3 |
| Relax (° C.) | 151 ± 3 |

The physical properties of these sutures were then tested utilizing standard tests known to those skilled in the art, the details of which are set forth below in Table 3.

TABLE 3
PROCEDURES FOR MEASURING PHYSICAL PROPERTIES
OF MONOFILAMENT SUTURES

| Physical Property | Test Procedure |
|---|---|
| knot-pull strength, kg | U.S.P. XXI tensile strength, sutures |
| | (881) |
| straight-pull strength, kg | ASTM D-2256, Instron Corporation |
| elongation, % | ATSM D-2256 |
| tensile strength (modulus), kg/mm² | ASTM D-2256, Instron Corporation |
| | Series IX Automated Materials |
| | Testing System 1.11 |

The results of these tests are set forth in Table 4 below.

TABLE 4

| | Straight | | Stress at | Modulus | Knot Pull |
|---|---|---|---|---|---|
| | Pull | Elongation | Max-Load | Average (Man | Average |
| | Average | Average | Average | Young) | Load at Max |
| Sample | (kg) | % | (Kg/mm²) | (Kg/mm²) | Load (kg) |
| 1 | 1.094 | 43.16 | 64.89 | 216.68 | .7722 |
| 2 | 1.103 | 44.51 | 65.23 | 209.95 | .7613 |
| 3 | 1.093 | 45.86 | 63.71 | 202.60 | .7668 |
| 4 | 1.059 | 46.33 | 65.04 | 207.20 | .7249 |
| 5 | 1.038 | 44.84 | 63.54 | 206.28 | .7187 |

### EXAMPLE 2

Additional samples from Example 1 were subjected to a sterilization step of ethylene oxide (EtO) and their physical properties were tested using the tests set forth in Table 3 above. The results of these tests are set forth in Table 5 below.

TABLE 5
STERILIZED SUTURES

| | | | | Modulus | Knot Pull |
|---|---|---|---|---|---|
| | Straight | | Stress at | Average | Average |
| | Pull | | Max-Load | (Man | Load at |
| | Average | Elongation | Average | Young) | Max Load |
| Sample | (kg) | Average % | (Kg/mm²) | (Kg/mm²) | (kg) |
| 1 | 1.130 | 38.74 | 67.52 | 274.1 | 0.7266 |
| 2 | 1.090 | 34.05 | 66.00 | 273.9 | 0.8177 |
| 3 | 1.125 | 39.86 | 68.15 | 273.7 | 0.8084 |
| 4 | 1.057 | 36.19 | 65.81 | 267.6 | 0.7958 |
| 5 | 1.068 | 38.55 | 65.57 | 265.75 | 0.7562 |

### EXAMPLE 3

Several studies were performed to evaluate the effects of varying the concentrations of distearylthiodipropionate (0.0%, 0.3% and 0.6%) in 5/0 polypropylene sutures with 0.75% polyethylene glycol distearate.

The sutures were sterilized prior to testing for fray resistance by exposure to EtO, at a temperature of 113° C., for 4 hours.

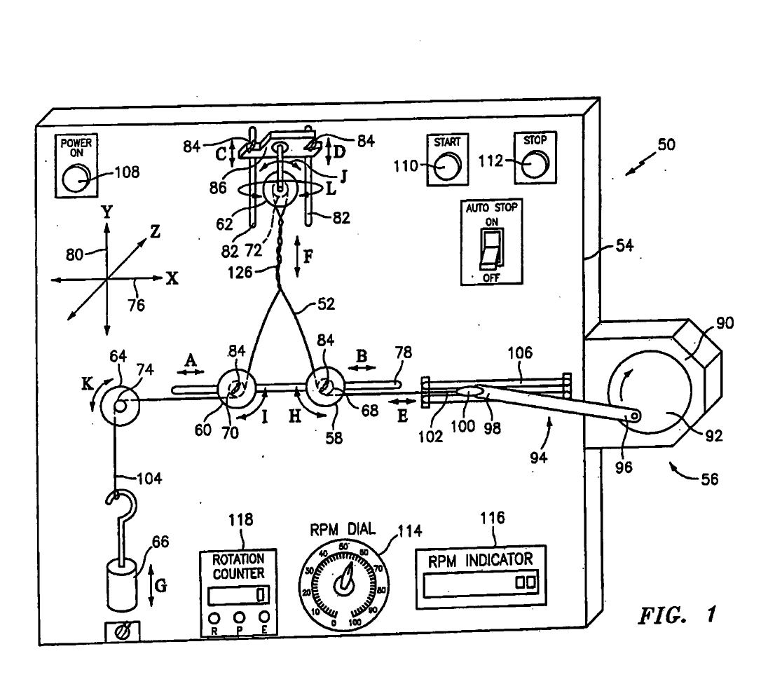

The various sterilized sutures were then exposed to a fray resistance test. The test measured the number of cycles needed to break (“CtB”) for each of the various propylene sutures. FIG. 1 illustrates the suture tester, utilized for measuring and evaluating fray resistance properties of the suture.

A suture was cut to an appropriate length. Referring to FIG. 1, a first end of the suture 102 is mounted to the suture gripper 100 of the suture tester 50. The suture 52 is then placed around the first pulley 58 and brought up vertically, making about an 80 degree wrap around the pulley. Then, the suture 52 is guided over the third pulley 62, making about a 300 degree wrap around the pulley. The suture 52 is then guided to the second pulley 60 and then brought over horizontally, making about an 80 degree wrap around the second pulley. Then, the suture 52 is guided over the fourth pulley 64 and brought down vertically, making about a 270 degree wrap around the fourth pulley. A tensioning element 66 is hung at the second end of the suture 104 using an adequate loop portion made thereof, to provide an adequate tension to the suture 52 for the test. Next, the third pulley 62 is rotated to create the desired number of suture wraps and the pulley 62 is then locked into place. The tensioning element was a fifty (50) gram weight for conventional size 5/0 sutures.

As shown in FIG. 1, turning the tester on results in reciprocating action to the suture's first end 102. This reciprocating action caused the suture's wrapped portion 126, and the tensioning element 66 to move substantially up and down as indicated by the doubled headed arrows F and G. In order to facilitate the reciprocating movement of the suture 52, the first, second, third and fourth pulleys 58, 60, 62 and 64 were subjected to subordinate rotational movement to back and forth directions as indicated by arrows H, I, J and K. Thus, the suture 52 rub against itself at its wrapped portion 126 while suitable tension is applied to the suture by the tensioning element 66.

Upon repeated rubbing action, the suture's wrapped portion 126 becomes fragile and eventually. Counter 118 displays the number of rubbing cycles or cycles to break at the time the suture fails.

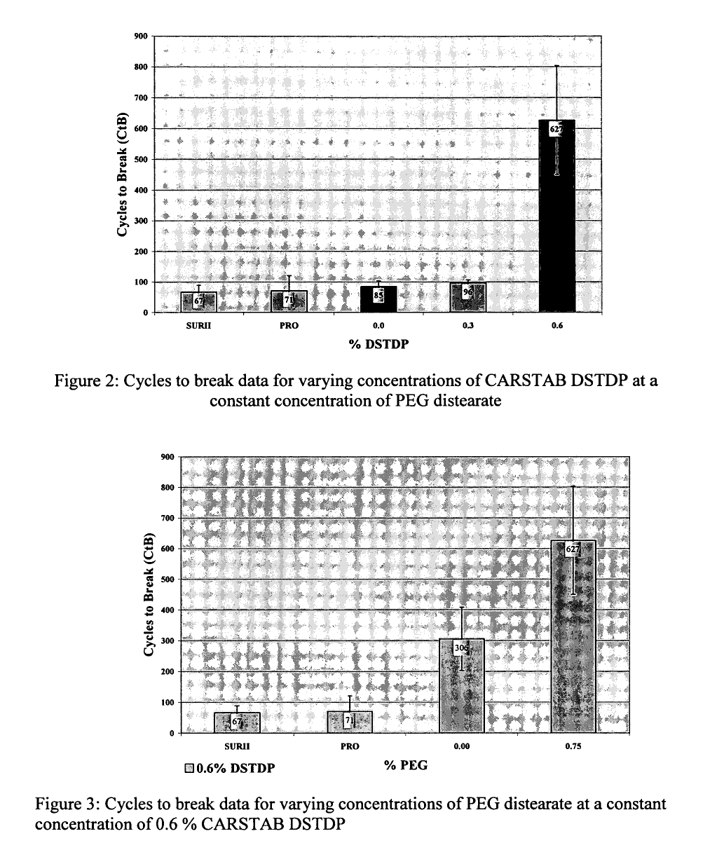

The results of these tests are set forth in FIG. 2. The fray testing showed a dramatic increase in cycles to break (CtB) with increased distearylthiodipropionate at a constant concentration of 0.75% PEG-distearate. Specifically, 85 CtB where no DSTDP was added compared to 630 CtB where 0.6% DSTDP was added. (See FIG. 2.)

### EXAMPLE 4

Sutures were prepared as in Example 3 above, with the difference being that in this Example the concentration of CARSTAB® DSTDP was held constant (0.6%) while the concentration of PEG distearate varied (0% and 0.75%). Fray testing was performed after sterilization as in Example 3, with the results set forth in FIG. 3. As can be seen in FIG. 3, an increase in CtB was found with increasing PEG distearate at a constant concentration of 0.6% CARSTAB® DSTDP (300 to 630 CtB).

As can be seen from the above, the addition of thiodipropionate enhanced the fray resistance of polyolefin sutures, with a thiodipropionate-polyolefin suture being more than three times more resistant to fraying than a polyolefin suture (300 CtB vs. 85 CtB) (See FIG. 3). Moreover, the addition of a fatty acid diester further enhanced fray resistance, with a thiodipropionate/fatty acid diester/polyolefin suture being more than two times more resistant to fraying than a thiodipropionate/polyolefin suture (630 CtB vs. 300 CtB) and more than seven times resistant to fraying than a polyolefin suture (630 CtB vs. 85 CtB).

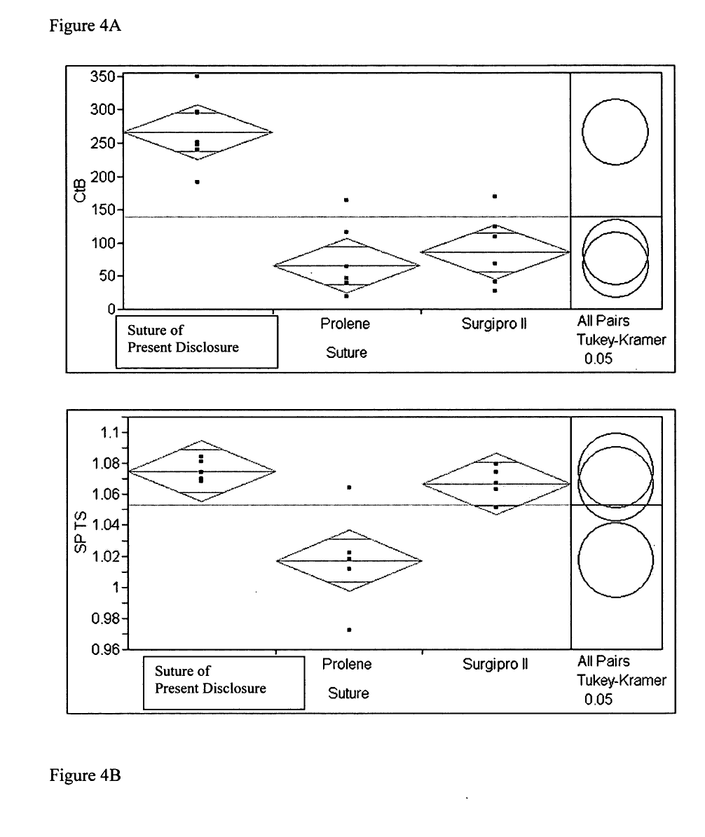

The sutures of the present disclosure were found to have enhanced pigment dispersion on visual examination. The fray resistance, cycles to break (CtB), and the tensile strength of the 5/0 sutures prepared in accordance with one embodiment of the present disclosure are graphically presented in FIGS. 4A and 4B. Differences in tensile strength and CtB were found when comparing the sutures of the present disclosure with the SURGIPRO II and PROLENE controls. The sutures of the present disclosure showed enhanced CtB compared with the SURGIPRO II and PROLENE sutures, without any sacrifice of their already superior tensile strength.

While the above description contains many specifics, these specifics should not be construed as limitations on the scope of the invention, but merely as exemplifications of particularly useful embodiments thereof. Those skilled in the art will envision many other possibilities within the scope and spirit of the invention as defined by the claims appended hereto.

What is claimed is:

1. A suture comprising: a sterilized filament, the filament comprising a composition containing a polyolefin and a fray-reducing amount of a thiodipropionate of the formula
R—OOCCH₂CH₂SCH₂CH₂COO—R
where R is a C₁₃ or greater alkyl group.

2. A suture as in claim 1 wherein the polyolefin is selected from the group consisting of polypropylene, polyethylene, copolymers of polyethylene and polypropylene and combinations thereof.

3. A suture as in claim 1 wherein the thiodipropionate is selected from the group consisting of distearylthiodipropionate, ditridecylthiodipropionate and combinations thereof.

4. A suture as in claim 1 wherein the thiodipropionate comprises from about 0.01% to about 1.5% by weight of the suture.

5. A suture as in claim 1 wherein the composition further comprises a fatty acid diester.

6. A suture as in claim 5 wherein the fatty acid diester is polyethylene glycol distearate.

7. A suture as in claim 7 wherein the fatty acid diester comprises from about 0.01% to about 5.0% by weight of the suture.

8. A suture as in claim 1 wherein the composition further comprises a synthetic wax.

9. A suture as in claim 8 wherein the synthetic wax is selected from the group consisting of polyethylene wax, ethylene copolymer wax, and halogenated hydrocarbon waxes.

10. A suture as in claim 8 wherein the synthetic wax comprises from about 0.01% to about 2.0% by weight of the suture.

11. A suture as in claim 1 wherein the composition further comprises a dye.

12. A suture as in claim 11 wherein the dye is selected from the group consisting of carbon black, bone black, copper phthalocyanine dyes, D&C Green No. 6, D&C Violet No. 2, indocyanine green, methylene blue, fluorescein, india ink, Prussian blue, eosins, acridine, iron oxide, and acramine yellow.

13. A suture as in claim 16 wherein the dye comprises from about 0.1% to about 1.0% by weight of the suture.

14. A suture as in claim 1 that is a monofilament suture.

15. A suture comprising polypropylene and a fray reducing amount of distearylthiodipropionate.

16. A method of fabricating a suture comprising:
a) providing a melt containing a polyolefin and a fray reducing amount of a thiodipropionate of the formula
R—OOCCH₂CH₂SCH₂CH₂COO—R
where R is a C₁₃ or greater alkyl group having at least 32 carbon atoms; and
b) extruding the melt to form a filament.

17. A method as in claim 16 wherein the step of providing a melt includes utilizing a polyolefin selected from the group consisting of polypropylene, polyethylene, copolymers of polyethylene and polypropylene, and combinations thereof.

18. A method as in claim 16 wherein the step of providing a melt includes utilizing a thiodipropionate selected from the group consisting of distearylthiodipropionate, ditridecylthiodipropionate and combinations thereof.

19. A method as in claim 16 wherein the step of providing the melt includes utilizing polyethylene glycol distearate as the polyalkylene glycol diester.

20. A method of suturing a wound comprising:
a) providing a needled suture having a suture as in claim 1; and
b) passing said needled suture through approximated wound tissue to create wound closure.

21. A method for fabricating a polyolefin suture comprising:
a) adding a thiodipropionate to a first portion of a polyolefin to form a masterbatch, where the thiodipropionate is of the formula
R—OOCCH₂CH₂SCH₂CH₂COO—R
and where R is a C₁₃ or greater alkyl group;
b) adding the masterbatch to a second portion of polyolefin to form a second batch;
c) heating the second batch to form a polyolefin melt; and
d) extruding the melt to form the polyolefin suture.

22. A method as in claim 21 wherein the polyolefin is selected from the group consisting of polypropylene, polyethylene, copolymers of polyethylene and polypropylene, and combinations thereof.

23. A method as in claim 21 wherein the polyolefin in the first portion is the same polyolefin as in the second portion.

24. A method as in claim 21 wherein the thiodipropionate is selected from the group consisting of distearylthiodipropionate, ditridecylthiodipropionate and combinations thereof.

25. A suture comprising:
a sterilized filament, the filament comprising a composition containing:
polypropylene;
distearylthiodipropionate in an amount from 0.01% to 1.5% based on the weight of the suture;
fatty acid diester in an amount from 0.01% to 5% based on the weight of the suture;
polyethylene wax in an amount from 0.01% to 2% based on the weight of the suture; and
optionally, a dye.
